(19) United States
(12) Patent Application Publication
(10) Pub. No.: US 2022/0330872 A1
(43) Pub. Date: Oct. 20, 2022

(54) BIO-SIGNAL MEASURING APPARATUS FOR DETECTING ABNORMAL SIGNAL SECTION IN ELECTROCARDIOGRAM DATA BY USING HEART SOUND DATA RELATED TO ELECTROCARDIOGRAM DATA, AND BIO-SIGNAL MEASURING METHOD

(71) Applicant: ATSENS CO., LTD., Gyeonggi-do (KR)

(72) Inventors: Jong Ook JEONG, Gyeonggi-do (KR); Chang Ho LEE, Gyeonggi-do (KR)

(73) Assignee: ATSENS CO., LTD., Gyeonggi-do (KR)

(21) Appl. No.: 17/232,938

(22) Filed: Apr. 16, 2021

(51) Int. Cl. A61B 5/33; A61B 5/00; A61B 5/333; A61B 7/04; A61B 5/346

(52) U.S. Cl. CPC A61B 5/33; A61B 5/7289; A61B 5/333; A61B 5/0006; A61B 7/04; A61B 5/346

(57) ABSTRACT

A bio-signal measuring apparatus includes a sensing apparatus configured to sense electrocardiogram data representing an electrical change according to a pulse of an object and sense heart sound data according to the pulse and a processing apparatus configured to store the electrocardiogram data in a memory. The processing apparatus is further configured to analyze the electrocardiogram data to determine whether or not an abnormal signal is generated in the electrocardiogram data, when the abnormal signal is detected to be generated in the electrocardiogram data, generate a storage control signal for heart sound data associated with the abnormal signal in an abnormal signal section including the abnormal signal, and store the associated heart sound data in the abnormal signal section of the memory in response to the storage control signal.

### BACKGROUND

### 1. Field

One or more embodiments relate to a bio-signal measuring apparatus for detecting an abnormal signal section in electrocardiogram data by using heart sound data associated with the electrocardiogram data, and a bio-signal measuring method.

### 2. Description of the Related Art

In order to maintain human life, there is a need for a process of enabling blood released by the heartbeat to flow along the arteries to all parts of the body without clogging and returning blood through the veins back to the heart. Accordingly, oxygen and nutrients may be supplied to the body's tissues, and consumed wastes may be removed through the metabolism.

However, when the human heart is in poor condition, blood may not be properly delivered to particular parts of the body or a blood clot or embolism may occur in the blood. As a result, blood may become cloudy, and the cloudy blood may block capillaries, in particular tissues of the body, and cause tissue necrosis, and thus, human life may be in danger. Therefore, in addition to clinical examinations, imaging tests and the like have been used to examine whether or not the heart is abnormal. Also, as an early diagnosis method, a method of determining whether or not a patient has an abnormality in the heart by measuring an electrocardiogram and displaying the measured electrocardiogram signal as a graph has also been widely used.

In other words, an electrocardiogram refers to recording of a potential change in the surface of the body according to the mechanical activity of the heartbeat, such as contraction or expansion of the heart muscle. The electrocardiogram is a non-vascular test that is simple to measure, easily reproduced, easily repetitively recorded, and inexpensive to test. The electrocardiogram has been used helpfully to diagnose arrhythmia and coronary artery disease (cardiac artery disease) and monitor the progress of cardiac patients.

In general, the electrocardiogram is measured by attaching a sensor for measuring an electrocardiogram on the upper left and right and lower left and right of the chest and using a potential difference detected according to the location of the sensor.

### SUMMARY

One or more embodiments according to the teachings of the present disclosure include a bio-signal measuring apparatus capable of simultaneously sensing electrocardiogram data and heart sound data, reducing a storage capacity of the heart sound data by allowing a point in time at which the heart sound data is stored to correspond to an abnormal signal section in the electrocardiogram data, and reducing communication resources for transmission of the heart sound data, and a bio-signal measuring method.

One or more embodiments include a bio-signal measuring apparatus for increasing the validity of an abnormal signal section generated in the heart by complementarily using electrocardiogram data and heart sound data, and a bio-signal measuring method.

Additional aspects will be set forth in part in the description which follows and, in part, will be apparent from the description, or may be learned by practice of the presented embodiments of the disclosure.

According to one or more embodiments, a bio-signal measuring apparatus capable of detecting an abnormal signal section in an electrocardiogram data by using heart sound data associated with the electrocardiogram data includes: a sensing apparatus configured to sense electrocardiogram data representing an electrical change according to a pulse of an object and sense heart sound data according to the pulse; and a processing apparatus configured to store the electrocardiogram data in a memory, analyze the electrocardiogram data to determine whether or not an abnormal signal is generated in the electrocardiogram data, when the abnormal signal is detected to be generated in the electrocardiogram data, generate a storage control signal for heart sound data in an abnormal signal section, and store the heart sound data in the abnormal signal section in the memory in response to the storage control signal.

In at least one variant, the processing apparatus may process the heart sound data in the abnormal signal section to transmit, to an external electronic device, the heart sound data in the abnormal signal section together with the electrocardiogram data and store, in the external electronic device, the heart sound data in the abnormal signal section together with electrocardiogram data.

In another variant, the processing apparatus may time-multiplex the electrocardiogram data and the heart sound data in the abnormal signal section stored in the memory.

In further another variant, the processing apparatus may sample the electrocardiogram data in the abnormal signal section at a first sampling rate, sample the heart sound data at a second sampling rate, tag corresponding points of the electrocardiogram data and the heart sound data in the abnormal signal section with time mark information, and determine validity of the abnormal signal section of the electrocardiogram data by using the heart sound data complementary to the electrocardiogram data.

In another variant, the processing apparatus may precisely synchronize the electrocardiogram data with the heart sound data by using first time mark information included in the electrocardiogram data at the first sampling rate and second time mark information included in the heart sound data at the second sampling rate.

In further another variant, the processing apparatus may determine the validity of the abnormal signal section by using the heart sound data complementary to the electrocardiogram data.

According to one or more embodiments, a bio-signal measuring apparatus includes a sensing apparatus configured to sense electrocardiogram data representing an electrical change according to a pulse of an object and sense heart sound data according to the pulse, and a processing apparatus configured to store the electrocardiogram data in a memory, transmit the electrocardiogram data to an external electronic device, and, when a storage control signal is received from the external electronic device, store heart sound data in an abnormal signal section in a memory of the external electronic device in response to the storage control signal.

According to one or more embodiments, a bio-signal measuring method includes sensing, by a bio-signal measuring apparatus, electrocardiogram data representing an electrical change according to a pulse of an object and sensing heart sound data according to the pulse, storing, by the bio-signal measuring apparatus, the electrocardiogram data in a memory, analyzing, by the bio-signal measuring apparatus, the electrocardiogram data to determine whether or not an abnormal signal is generated in the electrocardiogram data, when the bio-signal measuring apparatus detects that the abnormal signal is generated in the electrocardiogram data, generating, by the bio-signal measuring apparatus, a storage control signal for heart sound data in an abnormal signal section, and storing, by the bio-signal measuring apparatus, the heart sound data in the abnormal signal section in the memory in response to the storage control signal.

In at least one variant, the bio-signal measuring method may further include processing, by the bio-signal measuring apparatus, the heart sound data in the abnormal signal section to transmit, to an external electronic device, the heart sound data in the abnormal signal section together with the electrocardiogram data and store, in the external electronic device, the heart sound data in the abnormal signal section together with the electrocardiogram data.

In another variant, the determining may include time-multiplexing the electrocardiogram data and the heart sound data in the abnormal signal section stored in the memory.

In further another variant, the determining may include sampling the electrocardiogram data in the abnormal signal section at a first sampling rate, sampling the heart sound data at a second sampling rate, after the storing of the heart sound data in the memory, tagging corresponding points of the electrocardiogram data and the heart sound data in the abnormal signal section with time mark information and determining validity of the abnormal signal section of the electrocardiogram data by using the heart sound data complementary to the electrocardiogram data.

In another variant, the determining may include precisely synchronizing the electrocardiogram data with the heart sound data by using first time mark information included in the electrocardiogram data at the first sampling rate and second time mark information included in the heart sound data at the second sampling rate.

In yet another variant, the bio-signal measuring method may further include: after the storing of the heart sound data in the memory, determining the validity of the abnormal signal section by using the heart sound data complementary to the electrocardiogram data.

According to one or more embodiments, a computer program may be stored on a medium to execute, by using a computer, any one of methods according to one or more embodiments.

In addition, other methods and other systems for implementing one or more embodiments, and computer-readable recording media recording thereon a computer program for executing the method may be further included.

Other aspects, features, and advantages than those described above will become apparent from the following drawings, claims, and detailed description.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram of a bio-signal measuring apparatus according to one or more embodiments;

FIG. 2 is a block diagram illustrating transmission and reception of data between a sensing apparatus and a processing apparatus;

FIG. 3 is a block diagram of an analyzer of a processing apparatus;

FIG. 4 is a flowchart of a bio-signal measuring method according to one or more embodiments;

FIG. 5 is a view illustrating transmission and reception of data between a bio-signal measuring apparatus and a user terminal;

FIG. 6 is a flowchart of a bio-signal measuring method according to one or more embodiments;

FIG. 7 is a view for explaining a correlation between heart sound data and electrocardiogram data; and

FIG. 8 is a view for explaining an acquisition time interval in heart sound data.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

The expressions “comprises,” “includes,” “may comprise,” or “may include,” etc. that may be used in various embodiments indicate the presence of disclosed corresponding functions, operations, elements, or the like but do not limit additional at least one function, operation, element, or the like. Also, it will be understood that the terms “comprises,” “includes,” “have,” etc. when used herein, specify the presence of stated features, integers, steps, operations, elements, components, and/or combinations thereof but do not preclude the presence or addition of one or more other features, integers, steps, operations, elements, components, and/or combinations thereof.

In various embodiments, the expression “or” or the like includes any and all combinations of words listed together. For example, “A or B” may include A, may include B, or may include both A and B.

The expressions, such as “first”, “second” used in various embodiments, may modify various components of various embodiments but do not limit the corresponding components. For example, the above expressions do not limit the order and/or importance of the corresponding components. The above expressions may be used to distinguish one element or component from another element or component. For example, a first user device and a second user device are both user devices and represent different user devices. For example, a first element or component could be termed a second element or component, and, similarly, a second element or component could be termed a first element or component without departing from the scope of various embodiments.

When an element or component is referred to as being “coupled to” or “connected to” another element or component, it may be directly coupled to or connected to the other element or component, or intervening elements or components may be present. In contrast, when an element or component is referred to as being “directly coupled to” or “directly connected to” another element or component, intervening elements or components may not be present.

As used herein, the terms “module”, “unit”, “part”, etc. refers to an element or component that performs at least one function or operation, and these elements or components may be implemented as hardware, software, or a combination of hardware and software. In addition, “a plurality of modules”, “units”, “parts”, etc. may be integrated into at least one module or chip and implemented as at least one processor, except when each needs to be implemented as individual particular hardware.

The terminology used in various embodiments is for the purpose of describing particular embodiments only and is not intended to be limiting and/or restricting of various embodiments. The singular forms are intended to include the plural forms as well, unless the context clearly indicates otherwise.

Unless otherwise defined, all terms including technical or scientific terms used herein have the same meaning as commonly understood by one of ordinary skill in the art to which various embodiments belong.

It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, various embodiments will be described in detail with reference to the accompany drawings.

As used herein, a bio-signal refers to a signal including data such as body temperature, a pulse, an electrocardiogram, a brain wave, a respiratory rate, a step count, stress, hormones, exercise amount, burned calories, body fat, body water, a blood sugar value, blood pressure.

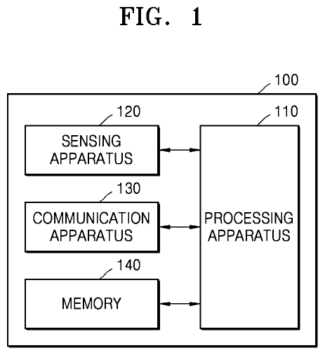

FIG. 1 is a block diagram of a bio-signal measuring apparatus 100 according to one or more embodiments.

The bio-signal measuring apparatus 100 may include a processing apparatus 110, a sensing apparatus 120, a communication apparatus 130, and a memory 140 to analyze an abnormal signal included in electrocardiogram data by complementarily using the electrocardiogram data and heart sound data.

The bio-signal measuring apparatus 100 may be an apparatus that measures a bio-signal of a human, an animal, or the like. The bio-signal measuring apparatus 100 may be non-invasively or invasively mounted on an object to measure an electrocardiogram according to a heartbeat of the object. The bio-signal measuring apparatus 100 may be implemented in a form attached to the skin or body of the object, but is not limited thereto and may be implemented in various ways. Here, the object may be a human, an animal, or a part of the body of the human or animal such as the chest, but is not limited thereto and may include all types of objects from which electrocardiograms may be detected or measured. In addition, an electrocardiogram refers to recording, in a graph, a potential change in the surface of the body according to the mechanical activity of the heartbeat, such as contraction/expansion of the heart muscle. The meaning of “detecting an electrocardiogram” is the same as that of “detecting an electric potential” generated in the surface of the body according to the heartbeat of an object.

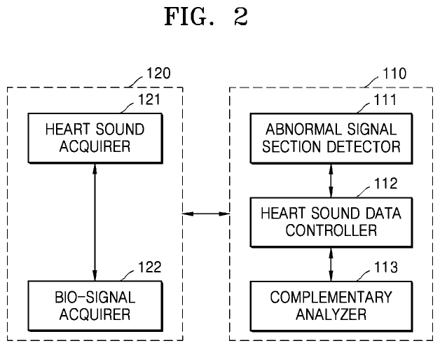

The processing apparatus 110 may be electrically connected to the sensing apparatus 120, the communication apparatus 130, and the memory 140 to process a bio-signal of an object. The processing apparatus 110 may receive electrocardiogram data and heart sound data sensed by the sensing apparatus 120 and analyze the electrocardiogram data by complementarily using the electrocardiogram data and the heart sound data. The processing apparatus 110 may detect an abnormal signal section in the electrocardiogram data on the basis of the heart sound data. The processing apparatus 110 may analyze the electrocardiogram data to detect information about the abnormal signal section. The processing apparatus 110 may determine the validity of the abnormal signal section by using the heart sound data corresponding to the abnormal signal section. The processing apparatus 110 may include detailed components as shown in FIG. 2.

The sensing apparatus 120 may be invasively or non-invasively attached to the body of an object to sense electrocardiogram data and/or heart sound data of the object. The sensing apparatus 120 may integrally or separately include a unit for sensing electrocardiogram data and/or a unit for sensing heart sound data. In some forms, the sensing apparatus 120 may measure electrocardiogram data of one or more channels via one or more electrodes. In other forms, the sensing apparatus 120 may receive electrocardiogram measurement data of one or more channels that is measured by one or more electrodes electrically connected. In one or more embodiments, the sensing apparatus 120 may be implemented to transmit electrocardiogram data and heart sound data via one or more lines. The sensing apparatus 120 may be implemented to divide time according to types of transmitted data, set a section for each piece of data, and transmit each piece of data in an allocated time interval to remote device.

The communication apparatus 130 is an apparatus for transmitting and receiving, via a communication network, data to and from an apparatus such as a server or another electronic device. The communication apparatus 130 is an apparatus for transmitting and receiving data via a wireless network, a wired network, or the like. The communication apparatus 130 may transmit and receive electrocardiogram data and/or heart sound data by processing the electrocardiogram data or heart sound data in a different method according to one or more control signals from the processing apparatus 110. Electrocardiogram data may be transmitted to an external electronic device. Heart sound data sensed by the sensing apparatus 120 may be processed so that only a portion thereof corresponding to an abnormal signal section selected in a control signal is transmitted to the external electronic device. Electrocardiogram data and/or heart sound data may be processed using one or more control signal from the processing apparatus 110.

The memory 140 may store a bio-signal including electrocardiogram data and/or heart sound data sensed by the sensing apparatus 120. The memory 140 may store a program for processing and controlling of the processing apparatus 110. The memory 140 may store data that is transmitted via the communication apparatus 130 and data that is received via the communication apparatus 130. The memory 140 may store electrocardiogram data generated by the processing apparatus 110, heart state information of an object, and the like. Electrocardiogram data and/or heart sound data may be stored in the memory 140. Electrocardiogram data and/or heart sound data may be processed so that only a portion thereof corresponding to an abnormal signal section selected in a control signal is stored in the memory 140. Here, the control signal is used to determine one or more method of processing measured data, to determine whether the measured data is stored, and to determine a storage location.

FIG. 1 illustrates that the processing apparatus 110 and the sensing apparatus 120 are provided in one apparatus, but the processing apparatus 110 and the sensing apparatus 120 may be provided and implemented in separate apparatuses. In this case, the processing apparatus 110 and the sensing apparatus 120 may be connected to each other electrically or via a communication network.

FIG. 2 is a block diagram for explaining an operation between the processing apparatus 110 and the sensing apparatus 120.

The sensing apparatus 120 may include a heart sound acquirer 121 and a bio-signal acquirer 122.

The heart sound acquirer 121 acquires a heart sound signal. In some forms, the heart sound acquirer 121 may be implemented by including a heart sound microphone for acquiring a heart sound and an AD conversion unit for converting a heart sound signal into heart sound data. The heart sound acquirer 121 may further include an amplification unit for amplifying a heart sound signal.

The bio-signal acquirer 122 may acquire an electrocardiogram signal that is an electrical change according to a heartbeat obtained by an electrode. The bio-signal acquirer 122 may include a measurement electrode for acquiring an electrocardiogram signal and an AD conversion unit for converting, into electrocardiogram data, the electrocardiogram signal measured by the measurement electrode. The bio-signal acquirer 122 may further include an amplification unit for amplifying the electrocardiogram signal.

Electrocardiogram data and heart sound data may be transmitted from the heart sound acquirer 121 and the bio-signal acquirer 122 to the processing apparatus 110 and processed by the processing apparatus 110. An abnormal signal section detector 111 may detect an abnormal signal section by analyzing electrocardiogram data.

The abnormal signal section detector 111 may analyze the electrocardiogram data and detect the abnormal signal section on the basis of a slope value, a peak value, or an R-R interval value. The abnormal signal section detector 111 may detect an R-peak candidate group on the basis of the slope value and an average value of peak values of the electrocardiogram data. The abnormal signal section detector 111 may detect the abnormal signal section from the R-peak candidate group on the basis of a peak average value, a slope average value, and an R-R interval value. The abnormal signal section detector 111 is not limited to the above-described embodiment and may detect the abnormal signal section on the basis of a voltage value, a pattern, and a slope value of the electrocardiogram data. Here, the pattern may include a morphological pattern in a time series arrangement of electrocardiogram data, a pattern between time values at which a peak occurs, a pattern between time values at which an inflection point occurs, and the like. Here, the slope value may be a slope value in a graph representing two-dimensional data of electrocardiogram data and voltage values. Here, an abnormal signal section refers to a time interval including a particular signal such as ventricular fibrillation, atrial fibrillation, or arrhythmia.

A heart sound data controller 112 may store, in a memory, heart sound data corresponding to the abnormal signal section in response to the storage control signal. The heart sound data controller 112 may transmit, to an external electronic device, heart sound data corresponding to an abnormal signal section in response to a storage control signal received from the external electronic device. The storage control signal may include information about the abnormal signal section. A complementary analyzer 113 may determine the validity of the abnormal signal section by complementing the heart sound data stored by the storage control signal.

When abnormal signal section detected by the abnormal signal section detection unit 111 coincides with the abnormal signal section in the heart sound data, the complementary analyzer 113 determines that an abnormal signal section is valid.

When the abnormal signal section does not coincide with the abnormal signal section in the heart sound data, the complementary analysis unit 113 may determine that the abnormal signal section detected by the abnormal signal section detection unit 111 is not valid. Through this, it can be determined that there has been an error in the measurement of the electrocardiogram data. If the abnormal signal section is longer than a given time interval, it may be determined that the biosignal acquisition unit 122 is not in a suitable state for measurement. For example, it may be determined that the biosignal acquisition unit 122 is not directly attached to the skin of the object and is spaced apart. When an abnormal signal section is not valid, the bio-signal acquirer 122 may not be directly attached to the skin of an object but may be spaced apart from the object. When an abnormal signal section is not valid, the bio-signal acquirer 122 may determine that electrocardiogram data includes noise.

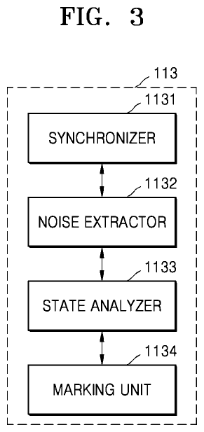

Detailed descriptions of components of the complementary analyzer 113, that is, a synchronizer 1131, a noise extractor 1132, a state analyzer 1133, and a marking unit 1134, will be given below with reference to FIG. 3.

The synchronizer 1131 may synchronize electrocardiogram data with heart sound data by complementarily using the electrocardiogram data and the heart sound data. The synchronization unit 1131 may synchronize the electrocardiogram data and the heart sound data based on a time value or an interval included in the electrocardiogram data and the heart sound data. The synchronizer 1131 may synchronize electrocardiogram data with heart sound data based on an abnormal signal section. In an abnormal signal section, the synchronizer 1131 may allow points of electrocardiogram data to respectively correspond to points of heart sound data determined by considering a heartbeat period determined at the points of the electrocardiogram data, thereby synchronizing the electrocardiogram data with the heart sound data. Here, the points of the electrocardiogram data may correspond to the points of the heart sound data determined by considering the heartbeat period. In this way, electrocardiogram data may be synchronized with heart sound data.

In one or more embodiments, points of electrocardiogram data and corresponding points of heart sound data may be tagged with pieces of time mark information associated with each other. The synchronizer 1131 may tag corresponding points of synchronized electrocardiogram data and heart sound data with pieces of time mark information associated with each other. In another embodiment, the synchronizer 1131 may tag electrocardiogram data and/or heart sound data with time mark information. Here, time mark information may be a recording time value recorded by the sensing apparatus 120, a reception time value received by the processing apparatus 110, or the like. Time mark information may be a time value or a time value within a time interval. In addition, time values may be tagged with respect to electrocardiogram data and/or heart sound data to correspond to points determined within a heartbeat period. For example, on a heartbeat period, electrocardiogram data and heart sound data may be tagged with time mark information for a peak point, a P wave point, a Q wave point, a starting point of the heartbeat period, and the like. In the heartbeat period, information about a peak point, a P wave point, a Q wave point, and a start point of the heart rate cycle may be converted into time mark information. The synchronizer 1131 may sample electrocardiogram data at a first sampling rate and sample heart sound data at a second sampling rate. The synchronizer 1131 may synchronize electrocardiogram data with heart sound data after tagging corresponding points of the electrocardiogram data and the heart sound data with time mark information. The synchronizer 1131 may synchronize electrocardiogram data with heart sound data by using first time mark information included in the electrocardiogram data at a first sampling rate and second time mark information included in the heart sound data at a second sampling rate. The first time mark information may include information corresponding to the second time mark information.

The synchronizer 1131 may synchronize a generation time of a 1-1^{th} point of electrocardiogram data and/or a generation time of a 1-2^{th} point of heart sound data. The 1-1^{th} point and the 1-2^{th} point may be corresponding points in a heartbeat section of an object. The synchronizer 1131 may synchronize electrocardiogram data and/or heart sound data by using marked time values.

In one or more embodiments, before synchronization, the synchronizer 1131 may further perform a process of removing noise from heart sound data. A noise removal algorithm for a sound, generally used in the relevant technical art, may be used for the process of removing noise from heart sound data but is not limited thereto.

In one or more embodiments, before synchronization, the synchronizer 1131 may time-multiplex electrocardiogram data and heart sound data. In a multiplexing process, electrocardiogram data may be sampled at a first sampling rate, and heart sound data may be sampled at a second sampling rate. The synchronizer 1131 may synchronize electrocardiogram data with heart sound data processed through the above process.

The synchronizer 1131 may determine the validity of an R peak of electrocardiogram data by using heart sound data after synchronizing the electrocardiogram data with the heart sound data. The validity of an R peak of electrocardiogram data may be determined by using heart sound data during a certain time interval from a generation point of the R peak. The heart sound has sound corresponding to the R peak.

The noise extractor 1132 may detect noise in electrocardiogram data by complementarily considering the electrocardiogram data and/or heart sound data. The noise extractor 1132 may extract noise in electrocardiogram data by using heart sound data complementary to the electrocardiogram data. Here, electrocardiogram data may include various types of noise, such as motion noise and muscle noise, due to signal characteristics.

In more detail, the noise extractor 1132 may detect noise of electrocardiogram data on the basis of a correlation between a pattern of changes in electrical components (voltage, current, and the like) of the electrocardiogram data and a frequency characteristic of heart sound data. The noise extractor 1132 may detect noise of heart sound data by using electrocardiogram data. The noise extractor 1132 may detect, in electrocardiogram data, signals having a low correlation with heart sound data and sections of the signals and detect, as noise, the corresponding signals and the sections of the corresponding signals. For example, when a 1-1^{th} point having a relatively highest voltage value in electrocardiogram data is detected as an unassociated value at a point 1-2^{th} of heart sound data corresponding to the 1-1^{th} point, electrocardiogram data at the corresponding 1-1^{th} point may be a signal having a low correlation with the heart sound data and may be detected as noise. The noise extractor 1132 may detect noise location information in which noise is generated, on the basis of a correlation between electrocardiogram data and heart sound data.

The noise extractor 1132 may determine, on the basis of generation point information of noise, the magnitude of noise in electrocardiogram data by comparing waveforms of electrocardiogram data at corresponding points in a previous period. Here, the magnitude of noise may be calculated as a difference value between data values of electrocardiogram data by comprising electrocardiogram data measured in a previous period with electrocardiogram data in a noise section on the basis of corresponding points. Here, the difference value may be a difference value between voltage values of pieces of electrocardiogram data.

The noise extractor 1132 may also detect noise included in heart sound data on the basis of a correlation with electrocardiogram data.

The state analyzer 1133 may generate heart state information of an object on the basis of electrocardiogram data and/or heart sound data processed via the synchronizer 1131 and the noise extractor 1132. The heart state information may include information corresponding to electrocardiogram data such as a heart rate, an R peak value, an R-R interval, and a P wave point, a form of the electrocardiogram data, disease information derived from a value, and the like.

The marking unit 1134 may mark, on electrocardiogram data or heart sound data, a tag corresponding to heart state information generated via the state analyzer 1133.

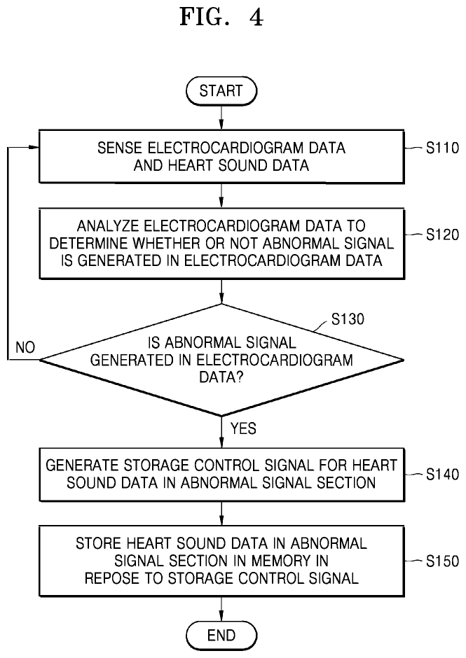

FIG. 4 is a flowchart of a bio-signal measuring method according to one or more embodiments.

As shown in FIG. 4, in operation S110, the bio-signal measuring apparatus 100 senses electrocardiogram data and heart sound data according to a pulse of an object. The bio-signal measuring apparatus 100 may integrally include a unit for sensing electrocardiogram data and heart sound data or may include a unit for sensing electrocardiogram data and a unit for sensing heart sound data, respectively.

In operation S120, the bio-signal measuring apparatus 100 may analyze the electrocardiogram data to determine whether or not an abnormal signal is generated in the electrocardiogram data.

The bio-signal measuring apparatus 100 may detect whether or not the abnormal signal is generated in the electrocardiogram data in operation S130 and generate a storage control signal for heart sound data in an abnormal signal section in operation S140. In operation S150, the bio-signal measuring apparatus 100 may store, in a memory, the heart sound data in the abnormal signal section in response to the storage control signal.

The bio-signal measuring apparatus 100 may store electrocardiogram data in the memory and, for heart sound data, may store, in the memory, only heart sound data corresponding to an abnormal signal section.

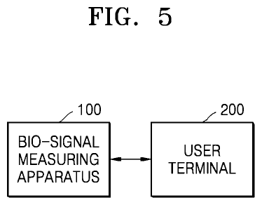

The bio-signal measuring apparatus 100 may transmit electrocardiogram data to an external electronic device and, for heart sound data, may transmit, to the external electronic device, only heart sound data corresponding to an abnormal signal section. FIG. 5 is a view for explaining transmission and reception of data between the bio-signal measuring apparatus 100 and a user terminal 200. Due to limitation of communication bandwidth, transmission and reception of the heart sound data corresponding to the abnormal signal section is usually necessary. And the received heart sound data will be used to re-process or re-check the abnormality at the user terminal 200. In another embodiment, the bio-signal measuring apparatus 100 may transmit the entire section of electrocardiogram data to the user terminal 200 and transmit an abnormal signal section of heart sound data to the user terminal 200. Electrocardiogram data and heart sound data may be converted into a standard format for transmission or storage.

Here, data corresponding to the abnormal signal section of the heart sound data may be determined by a storage control signal generated by the user terminal 200. The bio-signal measuring apparatus 100 may store heart sound data in an abnormal signal section in the memory in response to the storage control signal and transmit the heart sound data in the abnormal signal section to the user terminal 200. Accordingly, communication resources for transmission of heart sound data may be reduced.

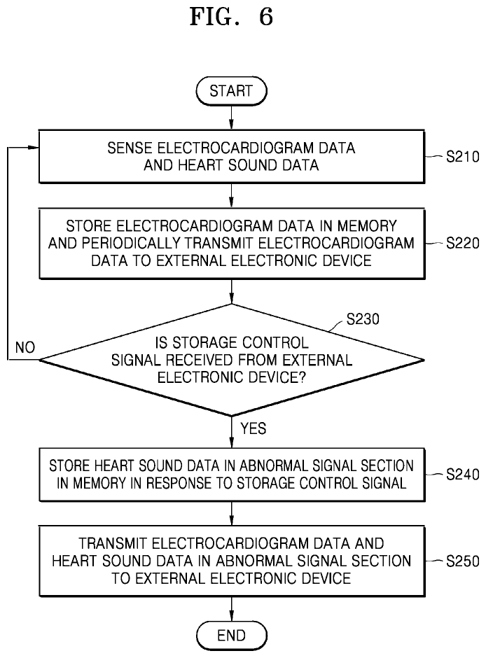

As shown in FIG. 6, in operation S210, the bio-signal measuring apparatus 100 periodically senses electrocardiogram data and heart sound data.

In operation S220, the bio-signal measuring apparatus 100 may store the electrocardiogram data in a memory and periodically transmit the electrocardiogram data to an external electronic device. And, the external electronic device detects an abnormal signal.

The bio-signal measuring apparatus 100 may detect, in operation S230, whether or not a storage control signal is received from the external electronic device and, in operation S240, store heart sound data in an abnormal signal section in the memory in response to the received storage control signal.

In operation S250, the bio-signal measuring apparatus 100 may transmit, to the external electronic device, electrocardiogram data and heart sound data in the abnormal signal section.

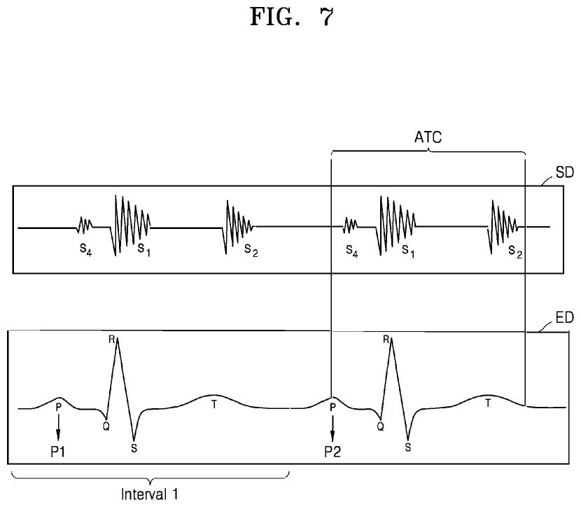

Heart sound data SD associated with P, Q, R, S, and T on a heartbeat interval 1 in electrocardiogram data ED representing an electrical change according to a pulse is generated as shown in FIG. 7.

Referring back to FIG. 2, the abnormal signal section detector 111 of the bio-signal measuring apparatus 100 may analyze the electrocardiogram data ED on the basis of a time interval (an R-R time interval, Interval 1, or the like) and detect an abnormal signal section of the electrocardiogram data ED on the basis of a waveform, a slope value, a peak value, and the like of the electrocardiogram data ED for each section. A time interval of electrocardiogram data may be determined by particular waves P1 and P2 included in the electrocardiogram data.

When an abnormal signal is detected even in heart sound data in an abnormal signal section, an abnormal signal section detected through electrocardiogram data may be determined to be valid.

When the abnormal signal is not detected in the heart sound data in the abnormal signal section, electrocardiogram data may be determined to be erroneously sensed in the abnormal signal section, due to detachment of a sensing unit, various types of noise during measurement of an electrocardiogram, for example, muscle noise, movement of an object, and the like.

For example, when a point P2 is determined to be an abnormal signal section, the bio-signal measuring apparatus 100 may generate a storage control signal, store heart sound data ATC corresponding to the abnormal signal section from among measured heart sound data, and process the heart sound data ATC to transmit the heart sound data ATC to an external electronic device. The bio-signal measuring apparatus 100 may determine the validity of an abnormal signal section by complementarily using electrocardiogram data and heart sound data.

In another embodiment, a storage control signal may be generated by an external electronic device and received. In this case, a function of detecting an abnormal signal section may be performed by the external electronic device. The external electronic device may determine the validity of the abnormal signal section by using heart sound data in the received abnormal signal section.

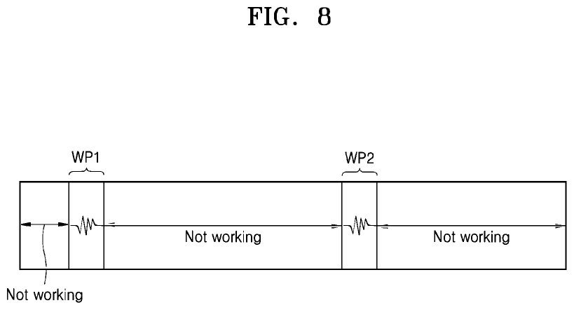

As shown in FIG. 8, the bio-signal measuring apparatus 100 according to one or more embodiments may be implemented to acquire heart sound data only in a particular time interval.

When the bio-signal measuring apparatus 100 extracts particular waves from electrocardiogram data, the bio-signal measuring apparatus 100 may acquire the particular waves only in particular time intervals WP1 and WP2 to complementarily use heart sound data. Here, the particular waves may be limited to abnormal signals or may refer to points that are periodically generated. A control signal for a heart sound acquisition period may be transmitted to the heart sound acquirer 121 of the bio-signal measuring apparatus 100, and the heart sound acquirer 121 may acquire a heart sound signal only in a particular time interval in the heart sound acquisition period according to the control signal. The particular time interval in which the heart sound signal is acquired may be determined via electrocardiogram data acquired through the bio-signal acquirer 122. The particular time interval in which the heart sound signal is acquired may be determined on the basis of a point in time at which a particular waveform of electrocardiogram is generated in electrocardiogram data, an abnormal signal section, and a point at which an abnormal signal is generated.

Apparatuses as described above may be implemented as hardware components, software components, and/or a combination of hardware components and software components. For example, apparatuses and components described in one or more embodiments may be implemented by using one or more general purpose computers or special purpose computers such as a processor, a controller, an arithmetic logic unit (ALU), a digital signal processor, a microcomputer, a field programmable gate array (FPGA), a programmable logic unit (PLU), a microprocessor, or any other apparatuses capable of executing and responding to instructions. A processing apparatus may execute an operating system (OS) and one or more software applications executed on the OS. In addition, the processing apparatus may access, store, manipulate, process, and generate data in response to the execution of software. For convenience of description, one processing apparatus may be described as being used, but one of ordinary skill in the art may understand that the processing apparatus may include a plurality of processing elements and/or a plurality of types of processing elements. For example, the processing apparatus may include a plurality of processors or one processor and one controller. Also, the processing apparatus may include another processing configuration such as a parallel processor.

Software may include a computer program, code, instructions, or a combination of one or more thereof, and configure the processing apparatus to operate as wanted or independently or collectively instruct the processing apparatus. Software and/or data may be permanently or temporarily embodied in any type of machine, a component, a physical device, virtual equipment, a computer storage medium or device, or transmitted signal waves to be interpreted by the processing apparatus or to provide the processing apparatus with instructions or data. The software may be distributed over networked computer systems to be stored or executed in a distributed manner. The software and data may be stored on one or more computer-readable recording media.

The method according to the embodiments may be embodied in the form of program instructions that may be executed through various types of computer means and then recorded on a computer-readable recording medium. The computer-readable recording medium may include program instructions, data files, data structures, and the like alone or in combination. The program instructions recorded on the computer-readable recording medium may be particularly designed and configured for the embodiments or may be well known to and used by one of ordinary skill in the computer software art. Examples of the computer-readable recording medium include magnetic media such as a hard disk, a floppy disk, and a magnetic tape, optical media such as a CD-ROM and a DVD, magneto-optical media such as a floptical disk, and a hardware device particularly configured to store and execute program instructions such as ROM, RAM, flash memory, and the like. Examples of the program instructions include not only machine language code generated by a compiler, but also high-level language code that may be executed by a computer by using an interpreter or the like. The hardware device described above may be configured to operate as one or more software modules so as to perform operations of the embodiments, and the reverse thereof is the same.

According to one or more embodiments, electrocardiogram data and heart sound data may be simultaneously sensed, and a point in time at which the heart sound data is stored may correspond to an abnormal signal section of the electrocardiogram data, thereby reducing a storage capacity of the heart sound data and reducing communication resources for transmission of the heart sound data.

In addition, the validity of the abnormal signal section generated in the heart may increase by complementarily using the electrocardiogram data and the heart sound data.

Although the embodiments have been described above by limited embodiments and drawings, various modifications and changes may be made from the above description by one of ordinary skill in the art. For example, the described techniques may be performed in a different order than the described method, and/or components of the described system, structure, device, circuit, and the like may be combined or joined in a different form than the described method, or even if replaced or substituted by other components or equivalents, and an appropriate result may be achieved.

Therefore, other embodiments, other aspects, and equivalents to the claims also belong to the scope of the claims that will be described below.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A bio-signal measuring apparatus for detecting an abnormal signal section in electrocardiogram data, comprising:
a sensing apparatus configured to sense electrocardiogram data representing an electrical change according to a pulse of an object and sense heart sound data according to the pulse; and
a processing apparatus configured to:
store the electrocardiogram data in a memory;
analyze the electrocardiogram data to determine whether or not an abnormal signal is generated in the electrocardiogram data;
upon detection of the abnormal signal in the electrocardiogram data, generate a storage control signal for first heart sound data associated with the abnormal signal in an abnormal signal section; and
store the first heart sound data in the abnormal signal section of the memory in response to the storage control signal.

2. The bio-signal measuring apparatus of claim 1, wherein the processing apparatus processes the first heart sound data to transmit, to an external electronic device, both the first heart sound data and the electrocardiogram data and store, in the external electronic device, the first heart sound data together with electrocardiogram data that are transmitted.

3. The bio-signal measuring apparatus of claim 1, wherein the processing apparatus time-multiplexes the electrocardiogram data and the first heart sound data.

4. The bio-signal measuring apparatus of claim 1, wherein the processing apparatus:
samples the electrocardiogram data in the abnormal signal section at a first sampling rate,
samples the first heart sound data stored in the abnormal signal section at a second sampling rate,
tags corresponding points of the electrocardiogram data and the heart sound data stored in the abnormal signal section with time mark information,
where the second sampling rate is different from a third sampling rate that samples second heart sound data stored in a normal signal section.

5. The bio-signal measuring apparatus of claim 4, wherein the third sampling rate is lower than the second sampling rate.

6. The external electronic device of claim 2, wherein the electrocardiogram data and the first heart sound data are converted into a standard format for transmission or storage.

7. The external electronic device of claim 6, wherein both the electrocardiogram data and the first heart sound data are stored for further analysis.

8. The external electronic device of claim 6, wherein upon verification that the first heart sound data is abnormal, the electrocardiogram data is stored and the first heart sound data is discarded.

9. A bio-signal measuring apparatus comprising:
a sensing apparatus configured to sense electrocardiogram data representing an electrical change according to a pulse of an object and sense heart sound data according to the pulse; and
a processing apparatus configured to:
store the electrocardiogram data in a memory,
transmit the electrocardiogram data to an external electronic device, and,
when a storage control signal is received from the external electronic device, store heart sound data associated with the storage control signal in an abnormal signal section in a memory of the external electronic device in response to the storage control signal.

10. A bio-signal measuring method comprising:
sensing, with a bio-signal measuring apparatus including a memory and a processor, electrocardiogram data representing an electrical change according to a pulse of an object and sensing heart sound data according to the pulse;
storing, with the bio-signal measuring apparatus, the electrocardiogram data in the memory;
analyzing, with the processor of the bio-signal measuring apparatus, the electrocardiogram data to determine whether or not an abnormal signal is generated in the electrocardiogram data;
when the bio-signal measuring apparatus detects that the abnormal signal is generated in the electrocardiogram data, generating, with the processor of the bio-signal measuring apparatus, a storage control signal for first heart sound data associated with the abnormal signal in an abnormal signal section; and
storing, by the bio-signal measuring apparatus, the first heart sound data in the abnormal signal section of the memory in response to the storage control signal.

11. The bio-signal measuring method of claim 10, further comprising:
processing, with the processor of the bio-signal measuring apparatus, the first heart sound data;
transmitting, to an external electronic device, the heart sound data stored in the abnormal signal section together with the electrocardiogram data; and
storing, in the external electronic device, both the heart sound data and the electrocardiogram data that are transmitted.

12. The bio-signal measuring method of claim 10, wherein determining includes time-multiplexing the electrocardiogram data and the first heart sound data in the abnormal signal section stored of memory.

13. The bio-signal measuring method of claim 10, wherein determining includes:
sampling the electrocardiogram data in the abnormal signal section at a first sampling rate,
sampling the first heart sound data at a second sampling rate,
subsequent to storing the first heart sound data in the memory, tagging corresponding points of the electrocardiogram data and the first heart sound data with time mark information, and
where the second sampling rate is different from a third sampling rate that samples second heart sound data stored in a normal signal section.

14. The bio-signal measuring method of claim 13, wherein the third sampling rate is lower than the second sampling rate.

15. A bio-signal processing method of claim 11, wherein the external electronic device is configured to convert the electrocardiogram data and the first heart sound data into a standard format for transmission or storage.
